(19) United States
(12) Patent Application Publication
(10) Pub. No.: US 2019/0355448 A1
(43) Pub. Date: Nov. 21, 2019

(54) AUTOMATED HEALTH ASSESSMENT SYSTEM AND METHOD THEREOF

(71) Applicant: SPOT CHECK MEDICAL SURGICAL EQUIPMENT & INSTRUMENTS TRADING, Business Bay Dubai (AE)

(72) Inventors: Manoj Kulangara MENON, Business Bay Dubai (AE); Manikandan Subramania PADMANABHA, Business Bay Dubai (AE); Santosh Kumar MENON, Business Bay Dubai (AE)

(73) Assignee: SPOT CHECK MEDICAL SURGICAL EQUIPMENT & INSTRUMENTS TRADING, Business Bay Dubai (AE)

(21) Appl. No.: 16/313,648

(22) PCT Filed: Jun. 20, 2017
(86) PCT No.: PCT/IB2017/053655

§ 371 Date: Dec. 27, 2018

(30) Foreign Application Priority Data
Jun. 28, 2016 (IN) 201641022121

(51) Int. Cl. G16H 10/60; G16H 50/30

(52) U.S. Cl. CPC G16H 10/60; G16H 80/00; G16H 50/30

(57) ABSTRACT

Embodiments of the present disclosure relates to a method and system for enabling automated health assessment. The method receives values of health related measurable parameters associated with a user and assigns a weightage score based on age and importance of the measurable parameters. The method also determines a parameter score corresponding to each parameter. Based on the weightage and parameter score, a health score of the user is determined for further recommendation of wellness programs suitable for the user. Thus, present disclosure enables measurement of a user's health related parameters and determines health score to indicate level of health thereby enabling a health coach to recommend one or more wellness programs suitable to the user for preventive health care. The system comprises a dedicated web-based and mobile-based application to effectively capture the measured information and constantly apprise and advise health status of the person at any point of time.

### TECHNICAL FIELD

The present subject matter is related, to health assessment kiosks in general and more particularly, but not exclusively to an automated health assessment system and method thereof.

### BACKGROUND

Hospitals, health clinics, and pharmacies, in addition to having an active role in supplying medical supplies and pharmaceuticals, have actively promoted various health care screenings and wellness programs. Health care assessment kiosks in hospitals, physician's offices, businesses, and the like, in combination with the growing number of home diagnostic kits that are available have increased the efficiencies in health care delivery. These kiosks use conventional technology to measure essential health parameters like blood pressure, heart rate and so on. However, these measurement kiosks are relatively large, expensive and time consuming, and require trained personnel to operate the kiosks. In addition to the above, the conventional kiosks do not provide any meaningful measurements of the measured health related parameters. The kiosk does not include any tools that help see a trend in the parameters in a timeline. Further, these kiosks do not provide any recommendations to the users to undergo wellness programs for preventive health care. Therefore, there is a need for a system and a method to automatically assess health of an individual or an organization, and overcoming the disadvantages and limitations of the existing systems.

### SUMMARY

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

Accordingly, the present disclosure relates to a method for enabling health assessment. The method includes receiving input values of a plurality of health related measurable parameters associated with a user, wherein the health related measurable parameters is one of parameter type including age dependant parameter and non-age dependant parameter. The method comprises assigning a weightage score to each of the age dependant and non-age dependant parameters and identifying a parameter score for each of the age dependant and non-age dependant parameter based on received input value of health related measurable parameter and predetermined range of values associated with each of the age dependant and non-age dependant parameter. Based on the parameter score and the weightage score corresponding to the age dependant and non-age dependant parameters, an overall health score indicative of health status of the user is determined.

Further, the disclosure relates to an automated health assessment device. The device comprises a processor and an input/output device coupled with the processor. The device also comprises at least a probe and a hand cuff assembly coupled with the processor. Further, the device comprises a memory communicatively coupled with the processor, wherein the memory stores processor-executable instructions, which, on execution, cause the processor to receive input values of a plurality of health related measurable parameters associated with a user, wherein the health related measurable parameter is one of parameter type including age dependant parameter and non-age dependant parameter. The processor further assigns a weightage score to each of the age dependant and non-age dependant parameters and identify a parameter score for each of the age dependant and non-age dependant parameter based on received input values of the health related measurable parameter and predetermined range of values associated with each of the age dependant and non-age dependant parameter. Based on the parameter score and the weightage score, the processor determines an overall health score indicative of health status of the user corresponding to the age dependant and non-age dependant parameters.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed embodiments. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates an exemplary architecture of a system for enabling automated health assessment in accordance with some embodiments of the present disclosure;

FIG. 2a illustrates an assessment chair enabling automated health assessment of a user in accordance with some embodiments of the present disclosure;

FIG. 2b illustrates an exemplary block diagram of automated health assessment device of FIG. 1 in accordance with some embodiments of the present disclosure;

FIG. 3 illustrates a flowchart of an exemplary method of enabling automated health assessment in accordance with some embodiments of the present disclosure; and

FIG. 4 is a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, “include(s)”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

Embodiments of the present disclosure relates to a method and a system for automated health assessment. The method receives values of health related measurable parameters associated with a user and assigns a weightage score based on age and importance of the measurable parameters. The method also determines a parameter score corresponding to each parameter. Based on the weightage and parameter score, a health score of the user is determined for further recommendation of wellness programs suitable for the user. Thus, present disclosure enables measurement of a user's health related parameters and determines health score to indicate level of health thereby enabling a health coach to recommend one or more wellness programs suitable to the user for preventive health care. The system comprises a dedicated web-based and mobile-based application to effectively capture the measured information and constantly apprise and advise health status of the person at any point of time. The system does not require any trained person to assist while conducting the health assessment and the complete analysis happens in less time without involving much complexity.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

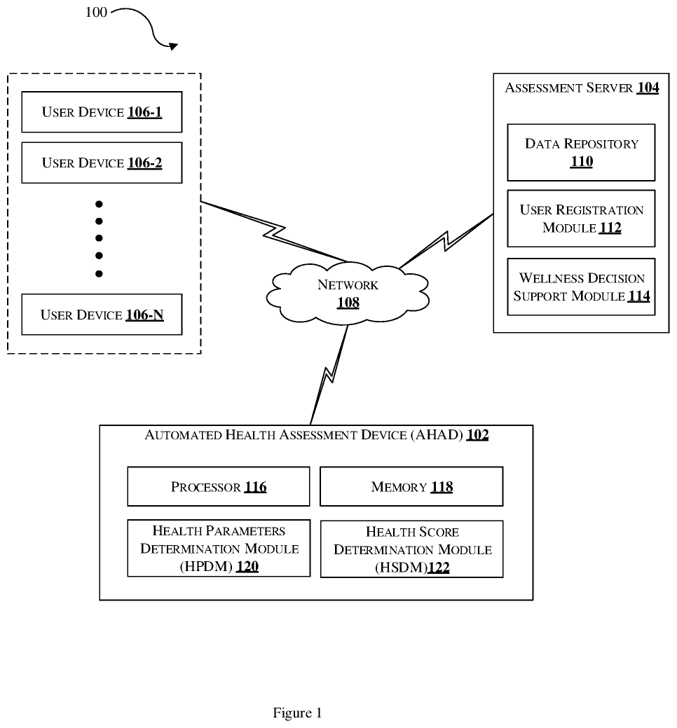

FIG. 1 illustrates an exemplary architecture of a system (100) for enabling automated health assessment in accordance with some embodiments of the present disclosure.

As shown in FIG. 1, the exemplary system (100) comprises one or more components configured for enabling automated health assessment. In one embodiment, the system (100) comprises an automated health assessment device (AHAD) (102), an assessment server (104) and one or more user devices (106-1), (106-2), (106-N) (hereinafter collectively referred to as user device (106)) communicatively coupled via a communication network (108). The communication network (108) may include, without limitation, a direct interconnection, local area network (LAN), wide area network (WAN), wireless network (e.g., using Wireless Application Protocol), the Internet, etc.

The AHAD (102) determines health status of users based on the health related measurable parameters and enables the assessment server (104) to recommend one or more wellness programs to the users via the user device (106) based on the health status. The user device (106) may be a mobile device or a computing device including the functionality for communicating over the network (108). For example, the mobile device can be a conventional web-enabled personal computer in the home, mobile computer (laptop, notebook or subnotebook), Smart Phone (iOS, Android & Windows), personal digital assistant, wireless electronic mail device, tablet computer or other device capable of communicating both ways over the Internet or other appropriate communications network. The assessment server (104) comprises a data repository (110) configured to store plurality of user profiles, one or more health related measurable parameters determined by AHAD (102) and plurality of health records associated with the one or more users. In one implementation, the data repository (110) may be integrated within the AHAD (102) independent of the wellness server (104). In another implementation, the data repository (110) may be configured independent of both the assessment server (104) and the AHAD (102) and communicatively connected via the network (108).

The assessment server (104) further comprises a user registration module (112) for enabling registration of a new user before the assessment process. In one embodiment, the user registration module (112) is configured to receive personal information like name, date of birth, gender, address, and contact information and so on from the users and create one or more user profiles associated with new users based on the received information. The user registration module (112) is further configured to assign unique user identification number (UID) to the new users for future references. In another embodiment, the user registration module (112) is capable of interfacing with available demographic databases for completing the registration process for the users associated with an organization.

Furthermore, the assessment server (104) comprises a wellness decision support module (WDSM) (114) configured to recommend one or more wellness programs to the one or more users via the user device (106) based on the health assessment results. In one embodiment, the WDSM (114) receives the health score and health status of the users from the AHAD (102) and provides recommendations or advice to be followed by the users based on the received health score and health status information. Upon determination, the WDSM (114) transmits one or more notifications related to the recommendations to the users via the user device (106) motivating the users to follow the advice and thereby improve the current health status to maintain a healthy life. In another embodiment, the WSDM (114) also determines one or more recommended wellness programs to the users via the user device (106) and transmits notifications about the one or more recommended wellness programs to the users at regular intervals. The WDSM (114) also updates the one or more user profiles of the users stored in the data repository (110) with the one or more recommended wellness programs associated with the users. Further, the WDSM (114) transmits the recommendations or general advice as well as the one or more recommended wellness programs to the AHAD (102) for inclusion in the report generated by the AHAD (102) for validation by a health coach or expert.

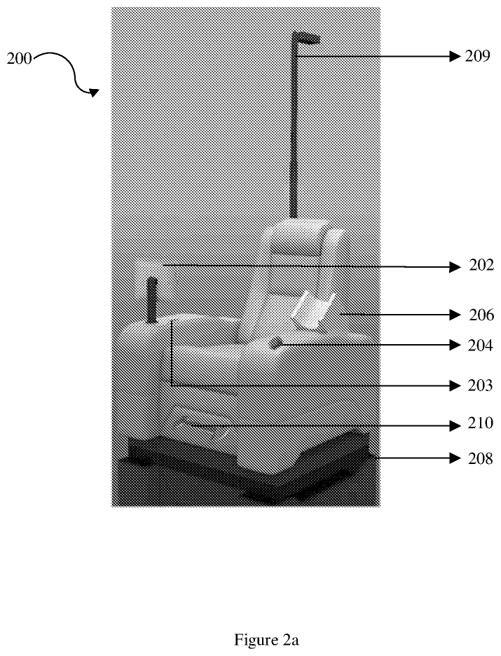

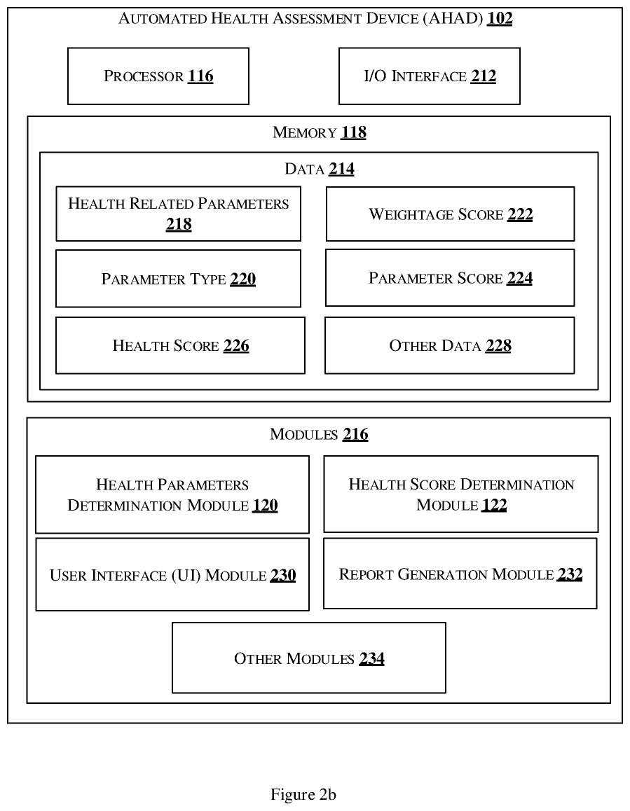

The AHAD (102) may be a typical automatic health assessment device. In one embodiment, the AHAD (102) comprises at least a processor (116), a memory (118) and an I/O interface communicatively coupled with the processor (116). The memory (118) stores data and modules and stores processor-readable instructions for performing the operations of the processor (116). The AHAD (102) also comprises a health parameters determination module (HPDM) (120), and a health score determination module (HSDM) (122). In one embodiment, the AHAD (102) may be configured within a body for example, a luxury chair seating (hereinafter referred to as “assessment chair”) (200) as illustrated in FIG. 2a. The assessment chair (200) facilitates the users to sit and conduct assessment of the health related measurable parameters associated with the users.

The assessment chair (200) comprises an Input/Output (I/O) device (202) communicatively coupled with the processor (116) and fixed onto a hand rest of the assessment chair (200). In one implementation, the I/O device (202) is configured to receive inputs via the I/O interface and transmit outputs for displaying in the I/O device (202) via the I/O interface. In one aspect, the AHAD (102) enables a new user to register and conduct health assessment for registered users. For example, the AHAD (102) enables a new user to register for health assessment via the I/O device (202). The I/O device (202) receives user's personal information like name, date of birth, gender, address, and contact information and so on for new user registration. The registered user may login into the web application on the AHAD (102) and continue with the health assessment. In another implementation, the user may provide his/her identity card over an ID card reader (203) that reads the identity card for user identification.

The assessment chair (200) further comprises a non-invasive probe (204) communicatively coupled with the processor (116) and the I/O device (202). The probe (204) is a non-invasive painless screening input point (probe) utilizing pulse wave technology to perform blood circulation analysis. For example, the probe (204) may be an infra-red light finger clip sensor that determines pulse wave of the user's blood from the time the pulse wave leaves the heart of the user and travels through the blood vessels down to the user's finger. The probe (204) is placed on another hand rest of the assessment chair (200) in which the user must insert his left index finger. The probe (204) is both a light emitter and detector that emits near infra-red rays to measure the pulse wave of the user. A pulse wave is generated when the heart pumps and a contour wave corresponding to the pulse wave is generated when the pulse wave travels along the artery tree of the heart. The processor (116) converts the contour wave thus generated into a corresponding digital signal and processes the digital signal to determine one or more health related measurable parameters such as blood circulation analysis.

The health related measurable parameters may include, but not limited to, complete body composition analysis, stress analysis and blood circulation analysis and so on. In one example, the complete body composition analysis includes one or more parameters like body weight, Body Mass Index (BMI), lean mass, fat tissue, bone mass, muscle protein mass, water content, fat free mass index, basic metabolic rate BMR, blood pressure BP, and resting heart rate and so on. In another example, the stress analysis includes Heart Rate Variability HRV tachogram, Mean Heart Rate, Artifact Beat, Balance of Sympathetic and Parasympathetic Nervous system, ANS—Autonomous Nervous System activity, Fatigue Index, Electro Cardiac Stability, Physical Stress, Mental Stress, Stress Resistance and Stress Score. In yet another example, the blood circulation analysis includes Pulse wave, Accelerated Pulse Wave Analysis, Vascular Health Analysis, Arterial Elasticity, Remaining blood volume and Wave Type.

The assessment chair (200) also comprises a hand cuff assembly (206) to automatically inflate and transfer the blood pressure measurement along with body fat measurements to the processor (116). The hand cuff assembly (206) comprises a cuff base and a flexible hand cuff attached to the cuff base. The cuff base is fixed onto the hand rest of the assessment chair (200) adjacent to the probe (204) and made of material like plastic. The hand cuff (206) is a flexible cuff member made of plastic or sturdy, flexible material or any combinations thereof to accommodate arms of user with varying diameters. To determine the blood pressure and body fat measurements, the user has to insert his/her arm into the hand cuff (206) such that the arm's biceps is sufficiently closer to the hand cuff (206) for proper inflation and measurement. The assessment chair (200) further comprises a weighing scale (208) positioned beneath the assessment chair (200) and communicatively coupled with the processor (116). The weighing scale (208) may be a distortion free weighing scale that ensures that a person can sit on the device for weight measurement to provide consistent accurate readings. The weighing scale (208) is concealed within the assessment chair (200) and not visible to the end user.

The assessment chair (200) also comprises a body composition analyser (not shown) communicatively coupled with the processor (116) and fixed beneath the assessment chair (200). The body composition analyser utilizes near-infrared technology to determine measurements of certain physical parameters of the users. In one embodiment, the body composition analyser measures the physical parameters in the body of the users and determines the composition from biceps or triceps using the near infrared technology. A probe is employed to emit and detect light projected onto the subject's body site i.e. triceps or biceps. The probe is placed inside the hand cuff (206) so that when the user inserts his/her arm into the hand cuff (206) upto the biceps/triceps, a detector located in the centre of the probe measures the amount of the light that is re-emitted from the biceps or triceps of the user. The processor (116) receives the detected signals from the detector and convert into equivalent absorbance units based on which determines measurements like blood pressure, body fat and weight. The probe of the body composition analyser emits harmless infrared light signals to take measurements and therefore do not suffer a loss in accuracy due to changes in body hydration or electrolyte levels. The body composition analyser device allows accurate percent fat measurements before, during or after exercising. Further, the processor (116) determines additional parameters like essential body fat, reserve body fat and excess body fat based on the percent fat measurements.

The assessment chair (200) also includes a height sensor (209) that uses ultrasonic waves to measure height of the person standing next to the assessment chair (200). The height sensor (209) is fixed behind the assessment chair (200) and communicatively coupled with the processor (116) to transfer the height measurement to the processor (116). Further, the assessment chair (200) also comprise a temperature sensor (not shown) disposed on the hand rest of the assessment chair (200) for measuring body temperature of the patient sitting on the assessment chair (200). In another embodiment, the body temperature of the patient can be determined by contactless thermometer using near infra-red ray technology. The assessment chair (200) is also provided with a foot rest (210) as illustrated in FIG. 2a.

In one embodiment, the AHAD (102) may be a typical AHAD as illustrated in FIG. 2b. The AHAD (102) comprises the processor (116), the memory (118), and an I/O interface (212). The I/O interface (202) is coupled with the processor (116) and the I/O device (202). The I/O device (202) is configured to receive inputs via the I/O interface (212) and transmit outputs for displaying in the I/O device (202) via the I/O interface (212). The I/O interface (212) may be configured to input user inputs required for customization and analysis of factors related to health of the organization.

The AHAD (102) further includes data (214) and modules (216). In one implementation, the data (214) may be stored within the memory (118). In one example, the data (214) may include health related parameters (218), parameter type (220), weightage score (222), parameter score (224), health score (226) and other data (228). In one embodiment, the data (214) may be stored in the memory (118) in the form of various data structures. Additionally, the aforementioned data can be organized using data models, such as relational or hierarchical data models. The other data (228) may be also referred to as reference repository for storing recommended implementation approaches as reference data. The other data (228) may also store data, including temporary data, temporary files and predetermined range values of parameter scores, predetermined range values of health parameters, suggested advice and recommended programs and health reports generated by the modules (216) for performing the various functions of the AHAD (102).

The modules (216) may include, for example, the HPDM (120), the HSDM module (122), a user interface (UI) module (230) and a report generation module (232). The modules (216) may also comprise other modules (234) to perform various miscellaneous functionalities of the AHAD (102). It will be appreciated that such aforementioned modules may be represented as a single module or a combination of different modules. The modules (216) may be implemented in the form of software performed by the processor, hardware and/or firmware.

In operation, the AHAD (102) enables measurement of health related measurable parameters (218) of user seated on the assessment chair (200), determines health score (226) of the user, and generates report indicating the health assessment results including the health score (226). In one embodiment, the UI module (230) enables the I/O device (202) to allow the user to input his/her credential information like name, user ID and so on. If the processor (116) determines that the user is a new user, the user registration module (112) enables creation of new user profile based on personal information received from the user via the I/O device (202) and stores the new user profile information in the data repository (110). If the processor (116) determines that the user has a valid user ID, then the I/O device (202) allows the user to enter his/her physiological information like for example, height and temperature information known to the user. In another embodiment, the IO device (202) transmits a signal to the processor (116) to automatically determine the physiological information and store the physiological information in the data repository (110). Upon validating the credential information of the user, the AHAD (102) continues the assessment process of the validated user.

In one embodiment, the HPDM (120) receives values of health related measurable parameters (218) like body composition analysis, stress analysis and blood circulation analysis based on the user's actions like his/her arm inserted upto the biceps/triceps within the hand cuff assembly (206), and his left index finger being inserted within the probe (204). The health related measurable parameters (218) include a plurality of age dependant parameters and a plurality of non-age dependent parameters. In one example, the plurality of age dependant parameters may include but not limited to, Arterial Elasticity (AE), Eccentric Constriction EC), remaining blood volume (RBV), systolic blood pressure, diastolic blood pressure, resting heart rate (RHR) and so on. In another example, the plurality of non-age dependant parameters may include but not limited to, BMI, excess fat, body fat percentage, stress score and vessel type. The HPDM (120) receives values of the health related measurable parameters (218) from the hand cuff assembly (206) and the probe (204) and determines the values of the age dependant and non-age dependant parameters.

In one embodiment, the HSDM (122) receives the values of the age dependant and non-age dependant parameters thus determined by the HSDM (122) and determine the health score based on the received parameter values. The HSDM (122) determines the health score based on one or more parameter score (224) for each of the determined parameters and a corresponding weightage score assigned to each of the determined parameters. In one implementation, the HSDM (122) determines the one or more parameter score (224) for the age dependant and non-age dependant parameters based on the measured parameter value, predetermined ranges of values and corresponding predetermined parameter rating. In one example, the predetermined ranges of values include an ideal or first value range, a second value range, a third value range and a worst value range.

In one exemplary embodiment, if the HSDM (122) determines the value of the health related measurable parameter (218) to fall in the ideal or first value range, then the corresponding parameter rating for example, ‘2’ is assigned to be the parameter score of the screening parameter. In another embodiment, if the HSDM (122) determines the value of the health related measurable parameter (218) to fall in the second value range, then the corresponding parameter rating for example, ‘4’ is assigned to be the parameter score of the screening parameter. In yet another embodiment, if the HSDM (122) determines the value of the health related measurable parameter (218) to fall in the third value range, then the corresponding parameter rating for example, ‘6’ is assigned to be the parameter score of the screening parameter. In another embodiment, if the HSDM (122) determines the value of the health related measurable parameter (218) to fall in the worst value range, then the corresponding parameter rating for example, ‘8’ is assigned to be the parameter score of the screening parameter. For non-age parameters like excess fat, vessel type and resting heart rate, the HSDM (122) assigns ‘1’ as the parameter score if the value of the health related measurable parameter (218) falls in the ideal or first range value. The HSDM (122) also determines a weightage score for each of the age dependant and non-age dependant parameters.

In one embodiment, the HSDM (122) detects the parameter type (220) of each of the health related measurable parameters (218) and assigns the weightage score (222) based on the parameter type (220). If the HSDM (122) determines the parameter type (220) to be age dependant parameter, then the HSDM (122) assigns the weightage score (222) to the age dependant parameters based on the age of the user. Age dependant parameters include for example, Arterial Elasticity (AE), Eccentric Constriction EC), remaining blood volume (RBV), systolic blood pressure, diastolic blood pressure, resting heart rate (RHR) based on the age of the user. For example, if the HSDM (122) determines the age of the user to fall in a first range for example 18-25, then the HSDM (122) assigns a first weightage score (222) of ‘2’ to the age dependant parameter. In another example, if the HSDM (122) determines the age of the user to fall in a second range for example 26-40, then the HSDM (122) assigns a second weightage score (222) of ‘4’ to the age dependant parameter. In yet another example, if the HSDM (122) determines the age of the user to fall in a third range for example 41-60, then the HSDM (122) assigns a third weightage score (222) of ‘6’ to the age dependant parameter. In still another example, if the HSDM (122) determines the age of the user to exceed more than 60, then the HSDM (122) assigns a fourth weightage score (222) of ‘8’ to the age dependant parameter. The HSDM (122) also assigns the weightage score (222) to the non-age dependant parameters based on importance of the parameter and a plurality of predetermined weightage scores.

In one embodiment, the HSDM (122) assigns the first weightage score (222) of ‘2’ to least important parameter like BMI, and assigns the weightage score (222) of ‘4’ to next important parameters like excess fat and body fat parameters. Further, the assigns the weightage score (222) of ‘8’ to most important parameters like stress and vessel type. Based on the assigned weightage score (222) and the parameter score (224), the HSDM (122) determines the health score of each of the age dependant and non-age dependant parameters. Upon determination of individual health score of each parameter, the HSDM (122) determines a final health score (226) of the user based on sum of all the individual health scores. Upon determining the final health score (226), the HSDM (122) determines a health status of the user and overall health score based on predetermined range values. For example, if the HSDM (122) determines the final health score (226) to fall in a first range of values like for example 1-163, then the HSDM (122) determines the health status to be “Excellent” with an AHAD health score 1′ assigned to the user. In another example, if the HSDM (122) determines the final health score (226) to fall in a second range of values like for example 164-254, then the HSDM (122) determines the health status to be “Good” with an AHAD health score ‘2’ assigned to the user. In yet another example, if the HSDM (122) determines the final health score (226) to fall in a third range of values like for example 255-344, then the HSDM (122) determines the health status to be “Careful” with an AHAD health score ‘3’ assigned to the user. In still another example, if the HSDM (122) determines the final health score (226) to fall in a fourth range of values like for example 345-1000, then the HSDM (122) determines the health status to be “Warning” with an AHAD health score ‘4’ assigned to the user.

The HSDM (122) also receives health risk information and medical history related information of the user as inputs through the I/O device (202). In one embodiment, the HRA and MH information may be stored as other data (228) in the AHAD (102). The user logins into the web portal or the mobile application using the I/O interface (212) and provides response to a plurality of queries related to health risk assessment (HRA) and medical history (MH) of the user. In one example, the plurality of queries relates to health risk assessment (HRA) which may be one or more of general health, food and exercise habits, medical risks and behaviour change related queries associated with the user. The plurality of queries of medical history may be, for example, associated with existing health condition of the user. The HSDM (122) optionally receives lab tests results provided by the user for health score determination.

The HSDM (122) receives the HRA and MH information in response to the plurality of queries associated with the HRA and MH and determines the health risk assessment score and medical history credit score respectively. The HSDM (122) uses the health risk assessment score and medical history credit score and the final health score (226) or the AHAD health score to determine the overall health score.

Based on the overall health score determined, the wellness program recommendation module (WDSM) (114) suggests one or more recommendations and recommended wellness programs to the users based on predetermined exposure of the overall health score. For example, if the WSDM (114) determines the overall health score to be ‘1’ (i.e. category 1), then the WDSM (114) recommends advice and one or more wellness programs for motivating to maintain a healthy life style. The advice for a user who is diabetic, may be for example instructions to walk for 30 minutes daily, check sugar levels daily, test for HBA1C for every 3 months and so on. In another case, if the user is having hypertension, the advice or recommendation would be to consume less intake of salt etc. In another example, if the WSDM (114) determines the overall health score to be ‘2’ (i.e. category 2), then the WDSM (114) recommends advice and one or more wellness programs to work on this category 2 and move to the category 1. In yet another example, if the WSDM (114) determines the overall health score to be ‘3’ (i.e. category 3), then the WDSM (114) recommends advice and one or more wellness programs to immediately alert and work on this category 3 to prevent future complications. In still another example, if the WSDM (114) determines the overall health score to be ‘4’ (i.e. category 4), then the WDSM (114) recommends advice and one or more wellness programs to immediately consult a physician for emergency treatment. Upon determination of the overall health score, one or more reports are generated with the evaluation results and stored in the assessment server (104).

In one embodiment, the report generation module (232) is configured to provide the one or more reports including the evaluation results with or without overall health score and suggested advice and recommended programs. The processor (116) transmits the generated reports to the user device (106) via SMS, Email and so on. In another embodiment, the generated reports are also referred to the health coach or experts for further opinion and alternate recommendations. Further, the processor (116) enables storing of the generated reports in the data repository (110) of the assessment server (104) for future references. Thus, the system (100) effectively captures the health status information and constantly apprises and advises health status of the person at any point of time. Further, the system (100) does not require any trained person to assist while conducting the test and the complete analysis happens in less time without involving much complexity.

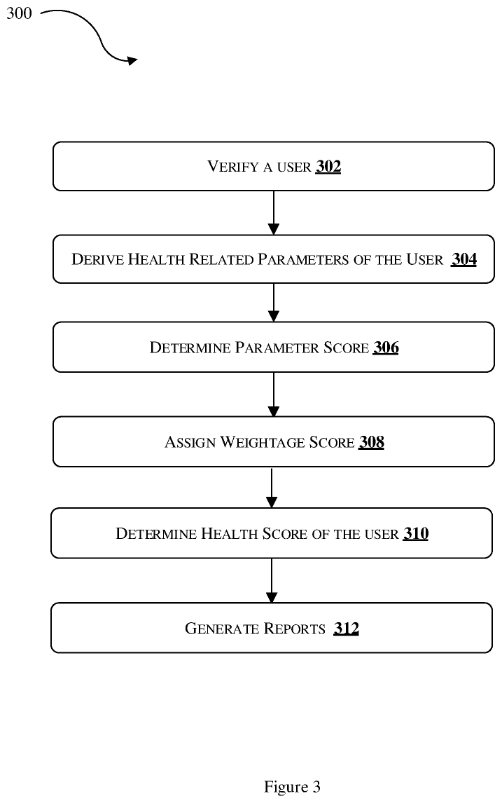

FIG. 3 illustrates a flowchart of an exemplary method of enabling automatic health assessment in accordance with some embodiments of the present disclosure; and

As illustrated in FIG. 3, the method (300) comprises one or more blocks implemented by the AHAD (102) for enabling automatic health assessment. The method (300) may be described in the general context of computer executable instructions. Generally, computer executable instructions can include routines, programs, objects, components, data structures, procedures, modules, and functions, which perform particular functions or implement particular abstract data types.

The order in which the method (300) is described is not intended to be construed as a limitation, and any number of the described method blocks can be combined in any order to implement the method (300). Additionally, individual blocks may be deleted from the method (300) without departing from the spirit and scope of the subject matter described herein. Furthermore, the method (300) can be implemented in any suitable hardware, software executed by a processor, firmware, or combination thereof.

At block (302), user is verified or authenticated. In one embodiment, the user seated on the assessment chair (200) is verified. In one embodiment, the I/O device (202) of the assessment chair (200) allows the user to enter his/her credential information like name, user ID and so on. If the processor (116) determines that the user is a new user, the user registration module (112) enables creation of new user profile based on personal information received from the user via the I/O device (202) and stores the new user profile information in the data repository (110). If the processor (116) determines that the user has a valid user ID, then the I/O device (202) allows the user to enter his/her physiological information known to the user. In another embodiment, the IO device (202) transmits a signal to the processor (116) to automatically determine the physiological information and store the physiological information in the data repository (110). Upon validating the credential information of the user, the AHAD (102) continues the screening process of the validated user.

At block (304), values of health related measurable parameters of the user are derived. In one embodiment, the HPDM (120) receives values of health related measurable parameters (218) like body composition analysis, stress analysis and blood circulation analysis based on the user's actions like his/her hand inserted within the hand cuff assembly (206), and his left index finger being inserted within the probe (204). The health related measurable parameters (218) include a plurality of age dependant parameters and a plurality of non-age dependent parameters. In one example, the plurality of age dependant parameters may include but not limited to, Arterial Elasticity (AE), Eccentric Constriction EC), remaining blood volume (RBV), systolic blood pressure, diastolic blood pressure, resting heart rate (RHR) and so on. In another example, the plurality of non-age dependant parameters may include but not limited to, BMI, excess fat, body fat percentage, stress score and vessel type. The HPDM (120) receives values of the health related measurable parameters (218) from the hand cuff assembly (206), the probe (204), the weighing scale (208) and the height sensor (209) and determines the health score of the user based on the received values.

At block (306), parameter score is determined. In one embodiment, the HSDM (122) receives the values of the health related measurable parameters (218) and determine the parameter score (224) based on the received parameter values. In one implementation, the HSDM (122) determines the one or more parameter score (224) for the age dependant and non-age dependant parameters based on the measured parameter value, predetermined ranges of values and corresponding predetermined parameter rating. In one example, the predetermined ranges of values include an ideal or first value range, a second value range, a third value range and a worst value range.

In one exemplary embodiment, if the HSDM (122) determines the value of the health related measurable parameter (218) to fall in the ideal or first value range, then the corresponding parameter rating for example, ‘2’ is assigned to be the parameter score of the screening parameter. In another embodiment, if the HSDM (122) determines the value of the health related measurable parameter (218) to fall in the second value range, then the corresponding parameter rating for example, ‘4’ is assigned to be the parameter score of the screening parameter. In yet another embodiment, if the HSDM (122) determines the value of the health related measurable parameter (218) to fall in the third value range, then the corresponding parameter rating for example, ‘6’ is assigned to be the parameter score of the screening parameter. In another embodiment, if the HSDM (122) determines the value of the health related measurable parameter (218) to fall in the worst value range, then the corresponding parameter rating for example, ‘8’ is assigned to be the parameter score of the screening parameter. For non-age parameters like excess fat, vessel type and resting heart rate, the HSDM (122) assigns ‘1’ as the parameter score if the value of the health related measurable parameter (218) falls in the ideal or first range value.

At block (308), weightage score of the health related measurable parameter is assigned. In one embodiment, the HSDM (122) also determines a weightage score for each of the age dependant and non-age dependant parameters. The HSDM (122) detects the parameter type (220) of each of the health related measurable parameters (218) and assigns the weightage score (222) based on the parameter type (220). If the HSDM (122) determines the parameter type (220) to be age dependant parameter, then the HSDM (122) assigns the weightage score (222) to the age dependant parameters based on the age of the user. For example, if the HSDM (122) determines the age of the user to fall in a first range for example 18-25, then the HSDM (122) assigns a first weightage score (222) of ‘2’ to the age dependant parameter. In another example, if the HSDM (122) determines the age of the user to fall in a second range for example 26-40, then the HSDM (122) assigns a second weightage score (222) of ‘4’ to the age dependant parameter. In yet another example, if the HSDM (122) determines the age of the user to fall in a third range for example 41-60, then the HSDM (122) assigns a third weightage score (222) of ‘6’ to the age dependant parameter. In still another example, if the HSDM (122) determines the age of the user to exceed more than 60, then the HSDM (122) assigns a fourth weightage score (222) of ‘8’ to the age dependant parameter.

The HSDM (122) also assigns the weightage score (222) to the non-age dependant parameters based on importance of the parameter and a plurality of predetermined weightage scores. In one embodiment, the HSDM (122) assigns the first weightage score (222) of ‘2’ to least important parameter like BMI, and assigns the weightage score (222) of ‘4’ to next important parameters like excess fat and body fat parameters. Further, the HSDM (122) assigns the weightage score (222) of ‘8’ to most important parameters like stress and vessel type.

At block (310), health score of the user is determined. In one embodiment, the HSDM (122) determines the health score of each of the age dependant and non-age dependant parameters based on the assigned weightage score (222) and the parameter score (224). Upon determination of individual health score of each parameter, the HSDM (122) determines a final health score (226) of the user based on sum of all the individual health scores.

The HSDM (122) also receives health risk information and medical history related information of the user as inputs through the I/O device (202). In one embodiment, the HRA and MH information may be stored as other data (228) in the AHAD (102). The user logins into the web portal or the mobile application using the I/O interface (212) and provides response to a plurality of queries related to health risk assessment (HRA) and medical history (MH) of the user. In one example, the plurality of queries relates to health risk assessment (HRA) which may be one or more of general health, food and exercise habits, medical risks and behaviour change related queries associated with the user. The plurality of queries of medical history may be, for example, associated with existing health condition of the user. The HSDM (122) optionally receives lab tests results provided by the user for health score determination.

The HSDM (122) receives the HRA and MH information in response to the plurality of queries associated with the HRA and MH and determines the health risk assessment score and medical history credit score respectively. The HSDM (122) uses the health risk assessment score and medical history credit score and the final health score (226) or the AHAD health score to determine the overall health score.

Based on the overall health score determined, the wellness program recommendation module (WDSM) (114) suggests one or more recommendations and recommended wellness programs to the users based on predetermined exposure of the overall health score. For example, if the WSDM (114) determines the overall health score to be ‘1’ (i.e. category 1), then the WDSM (114) recommends advice and one or more wellness programs for motivating to maintain a healthy life style. The advice for a user who is diabetic, may be for example instructions to walk for 30 minutes daily, check sugar levels daily, test for HBA1C for every 3 months and so on. In another case, if the user is having hypertension, the advice or recommendation would be to consume less intake of salt etc. In another example, if the WSDM (114) determines the overall health score to be ‘2’ (i.e. category 2), then the WDSM (114) recommends advice and one or more wellness programs to work on this category 2 and move to the category 1. In yet another example, if the WSDM (114) determines the overall health score to be ‘3’ (i.e. category 3), then the WDSM (114) recommends advice and one or more wellness programs to immediately alert and work on this category 3 to prevent future complications. In still another example, if the WSDM (114) determines the overall health score (to be ‘4’ (i.e. category 4), then the WDSM (114) recommends advice and one or more wellness programs to immediately consult a physician for emergency treatment. Upon determination of the overall health score, one or more reports are generated with the evaluation results and stored in the assessment server (104).

At block (312), reports are generated. In one embodiment, the report generation module (232) is configured to provide the one or more reports including the evaluation results with or without overall health score and suggested advice and recommended programs. The processor (116) transmits the generated reports to the user device (106) via SMS, Email, Mobile App, Web Portal and so on. In another embodiment, the generated reports are also referred to the health coach or experts for further opinion and alternate recommendations. Further, the processor (116) enables storing of the generated reports in the data repository (110) of the assessment server (104) for future references.

Thus, the system (100) effectively captures the health status information and constantly apprises and advises health status of the person at any point of time. Further, the system (100) does not require any trained person to assist while conducting the test and the complete analysis happens in less time without involving much complexity.

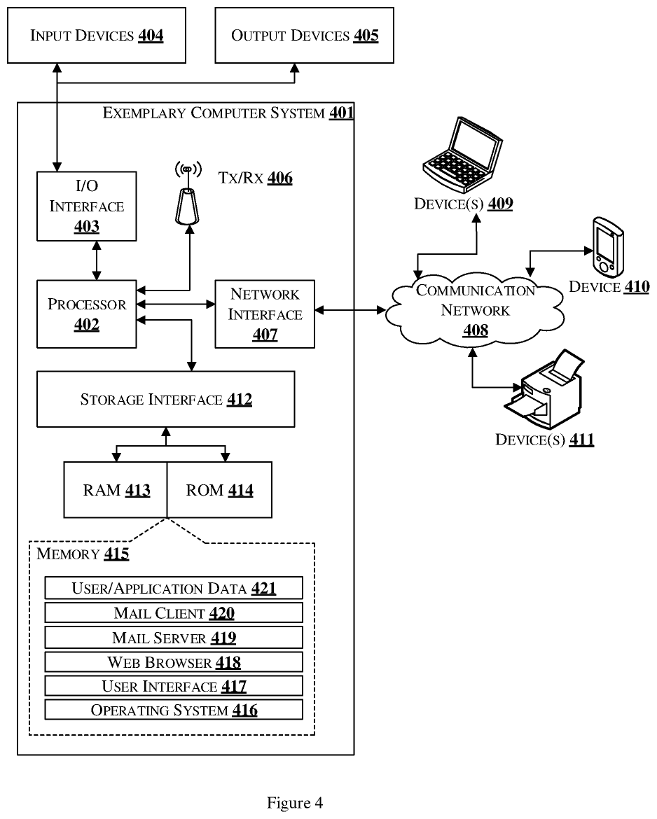

FIG. 4 is a block diagram of an exemplary computer system for implementing embodiments consistent with the present disclosure.

Variations of computer system (401) may be used for implementing all the computing systems that may be utilized to implement the features of the present disclosure. Computer system (401) may comprise a central processing unit (“CPU” or “processor”) (402). The processor (402) may comprise at least one data processor for executing program components for executing user- or system-generated requests. The processor 402 may include specialized processing units such as integrated system (bus) controllers, memory management control units, floating point units, graphics processing units, digital signal processing units, etc. The processor (402) may include a microprocessor, such as AMD Athlon, Duron or Opteron, ARM's application, embedded or secure processors, IBM PowerPC, Intel's Core, Itanium, Xeon, Celeron or other line of processors, etc. The processor (402) may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor (402) may be disposed in communication with one or more input/output (I/O) devices via I/O interface (403). The I/O interface (403) may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface (403), the computer system (401) may communicate with one or more I/O devices. For example, the input device (404) may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. Output device 405 may be a printer, fax machine, video display (e.g., cathode ray tube (CRT), liquid crystal display (LCD), light-emitting diode (LED), plasma, or the like), audio speaker, etc. In some embodiments, a transceiver (406) may be disposed in connection with the processor (402). The transceiver (406) may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, the processor (402) may be disposed in communication with a communication network (408) via a network interface (407). The network interface (407) may communicate with the communication network (408). The network interface (407) may employ connection protocols including, without limitation, direct connect, Ethernet (e.g., twisted pair 10/40/400 Base T), transmission control protocol/internet protocol (TCP/IP), token ring, IEEE 802.11a/b/g/n/x, etc. The communication network (408) may include, without limitation, a direct interconnection, local area network (LAN), wide area network (WAN), wireless network (e.g., using Wireless Application Protocol), the Internet, etc. Using the network interface (407) and the communication network (408), the computer system (401) may communicate with devices (409), (410), and (411). These devices (409), (410), and (411) may include, without limitation, personal computer(s), server(s), fax machines, printers, scanners, various mobile devices such as cellular telephones, smartphones (e.g., Apple iPhone, Blackberry, Android-based phones, windows based phones etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system 401 may itself embody one or more of these devices.

In some embodiments, the processor (402) may be disposed in communication with one or more memory devices (e.g., RAM (413), ROM (414), etc.) via a storage interface (412). The storage interface (412) may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

The memory (415) may store a collection of program or database components, including, without limitation, an operating system (416), a user interface application (417), a web browser (418), a mail server (419), a mail client (420), user/application data (421) (e.g., any data variables or data records discussed in this disclosure), etc. The operating system (416) may facilitate resource management and operation of the computer system (401). Examples of the operating system (416) include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. The user interface application (417) may facilitate display, execution, interaction, manipulation, or operation of program components through textual or graphical facilities. For example, user interfaces may provide computer interaction interface elements on a display system operatively connected to the computer system (401), such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, the computer system (401) may implement a web browser (418) stored program components. The web browser (418) may be a hypertext viewing application, such as Microsoft Internet Explorer, Google Chrome, Mozilla Firefox, Apple Safari, etc. Secure web browsing may be provided using HTTPS (secure hypertext transport protocol), secure sockets layer (SSL), Transport Layer Security (TLS), etc. The web browser (418) may utilize facilities such as AJAX, DHTML, Adobe Flash, JavaScript, Java, application programming interfaces (APIs), etc. In some embodiments, the computer system (401) may implement a mail server (419) stored program components. The mail server (419) may be an Internet mail server such as Microsoft Exchange, or the like. The mail server (419) may utilize facilities such as ASP, ActiveX, ANSI C++/C#, Microsoft .NET, CGI scripts, Java, JavaScript, PERL, PHP, Python, WebObjects, etc. The mail server (419) may utilize communication protocols such as internet message access protocol (IMAP), messaging application programming interface (MAPI), Microsoft Exchange, post office protocol (POP), simple mail transfer protocol (SMTP), or the like. In some embodiments, the computer system (401) may implement a mail client (420) stored program components. The mail client (420) may be a mail viewing application, such as Apple Mail, Microsoft Entourage, Microsoft Outlook, Mozilla Thunderbird, etc.

In some embodiments, computer system (401) may store user/application data (421), such as the data, variables, records, etc. as described in this disclosure. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of the any computer or database component may be combined, consolidated, or distributed in any working combination.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory.

Examples include random access memory (RAM), read-only memory (ROM), volatile memory, nonvolatile memory, hard drives, CD ROMs, DVDs, flash drives, disks, and any other known physical storage media.

### Advantages of the Embodiment of the Present Disclosure are Illustrated Herein

In an embodiment, the present disclosure enables health assessment of a user by based on age dependent parameters and important non-age dependent parameters.

In an embodiment, the present disclosure captures the health status information and constantly apprises and advises health status of the person at any point of time.

In another embodiment, the system does not require any trained person to assist while conducting the test and the complete analysis happens in less time without involving much complexity.

It is intended that the disclosure and examples be considered as exemplary only, with a true scope and spirit of disclosed embodiments being indicated by the following claims.

We claim:

1. A method for health assessment, method comprising:
receiving, by a processor (116) of a health assessment device (102), input values of a plurality of health related measurable parameters (218) associated with a user, wherein the health related measurable parameters (218) is one of parameter type (220) including age dependant parameter and non-age dependant parameter;
assigning, by the processor (116), a weightage score (222) to each of the age dependant and non-age dependant parameters;
identifying, by the processor (116), a parameter score (224) for each of the age dependant and non-age dependant parameter based on received input value of health related measurable parameter (218) and predetermined range of values associated with each of the age dependant and non-age dependant parameter; and
determining, by the processor (116), a final health score (226) indicative of health status of the user based on the parameter score (224) and the weightage score (222) corresponding to the age dependant and non-age dependant parameters.

2. The method as claimed in claim 1, wherein assigning the weightage score (222) to each of the age dependant and non-age dependant parameters, comprising:
detecting the parameter type (220) of each of the health related measurable parameters (218) received as input;
assigning one of a first, second, third and fourth weightage score (222) to the health related measurable parameter (218) based on age of the user upon detection of the parameter type (220) to be age dependant parameter; and
assigning one of the first, second and fourth weightage score (222) to the non-age dependant parameter upon detection of the parameter type (220) to be non-age dependant parameter.

3. The method as claimed in claim 2, where assigning one of the first, second and third weightage score (222) comprising assigning weightage score (222) in increasing order to the age dependant parameter proportional to increase in the age of the user.

4. The method as claimed in claim 2, where assigning one of the first, second and fourth weightage score (222) comprising assigning the weightage score (222) to the non-age dependant parameter based on the importance of the non-age dependant parameter.

5. The method as claimed in claim 1, wherein the age dependant parameter is selected from group comprising Arterial Elasticity (AE), Eccentric Constriction EC), remaining blood volume (RBV), systolic blood pressure, diastolic blood pressure, resting heart rate (RHR) and other age dependant parameters.

6. The method as claimed in claim 1, wherein the non-age dependant parameter is selected from group comprising body mass index (BMI), excess fat, body fat percentage, stress score and vessel type and other non-age dependant parameters.

7. The method as claimed in claim 1, further comprising:
determining overall health score based on the final health score (226), health risk assessment and medical history related information;
evaluating a health status information corresponding to the overall health score, wherein the health status information is indicative of the health status of the user; and
determining one or more recommendations and recommended wellness program based on the health status information and the overall health score.

8. The method as claimed in claim 1, further comprising:
determining overall health score of an organization based on the overall health score of all users associated with the organization.

9. An automated health assessment device (102), comprising:
a processor (116) and an input/output device coupled with the processor (116);
at least a probe (204) and a hand cuff assembly (206) coupled with the processor (116); and
a memory (118) communicatively coupled with the processor (116), wherein the memory (118) stores processor-executable instructions, which, on execution, cause the processor (116) to:
receive input values of a plurality of health related measurable parameters (218) associated with a user, wherein the health related measurable parameter (218) is one of parameter type (220) including age dependant parameter and non-age dependant parameter;
assign a weightage score (222) to each of the age dependant and non-age dependant parameters;
identify a parameter score (224) for each of the age dependant and non-age dependant parameter based on received input values of the health related measurable parameter (218) and predetermined range of values associated with each of the age dependant and non-age dependant parameter; and
determine a final health score (226) indicative of health status of the user based on the parameter score (224) and the weightage score (222) corresponding to the age dependant and non-age dependant parameters.

10. The device as claimed in claim 9, wherein the processor (116) is configured to assign the weightage score (222) to each of the age dependant and non-age dependant parameters, by steps of:
detecting the parameter type (220) of each of the health related measurable parameters (218) received as input;
assigning one of a first, second, third and fourth weightage score (222) to the health related measurable parameter (218) based on age of the user upon detection of the parameter type (220) to be age dependant parameter; and
assigning one of the first, second and fourth weightage score (222) to the non-age dependant parameter upon detection of the parameter type (220) to be non-age dependant parameter.

11. The device as claimed in claim 10, where the processor (116) assigns one of the first, second and third weightage score (222) in increasing order to the age dependant parameter proportional to increase in the age of the user.

12. The device as claimed in claim 10, where processor (116) assigns one of the first, second and fourth weightage score (222) based on the importance of the non-age dependant parameter.

13. The device as claimed in claim 9, wherein the age dependant parameter is selected from group comprising Arterial Elasticity (AE), Eccentric Constriction EC), remaining blood volume (RBV), systolic blood pressure, diastolic blood pressure, resting heart rate (RHR) and other age dependant parameters.

14. The device as claimed in claim 9, wherein the non-age dependant parameter is selected from group comprising body mass index (BMI), excess fat, body fat percentage, stress score and vessel type and other non-age dependant parameters.

15. The device as claimed in claim 9, wherein the processor (116) is configured to receive a pulse wave signal from the probe (204) and convert into a corresponding digital signal for determination of health related measurable parameters (218).

16. The device as claimed in claim 9, wherein the probe (204) is a non-invasive infra-red light finger clip sensor attached to the left index finger of the user and configured to generate the pulse wave signal indicative of the pulse wave of the user.

17. The device as claimed in claim 9, wherein the hand cuff assembly (206) comprises a cuff base and a flexible hand cuff attached to the cuff base, wherein the cuff base is fixed adjacent to the probe (204) onto the hand rest of the device (102).

18. The device as claimed in claim 9, wherein the hand cuff assembly (206) is configured to automatically inflate when the user's hand is inserted and determine blood pressure and body fat measurement values to the processor (116).

19. The device as claimed in claim 9, wherein the processor (116) is further configured to:
determine overall health score based on the final health score (226), health risk assessment and medical history related information;
evaluate a health status information corresponding to the overall health score, wherein the health status information is indicative of the health status of the user; and
determine one or more recommendations and recommended wellness program based on the health status information and the overall health score.

20. The device as claimed in claim 9, wherein the processor (116) is further configured to determine overall health score of an organization based on the overall health score of all users associated with the organization.
